(19) United States
(12) Patent Application Publication
(10) Pub. No.: US 2009/0242495 A1
(43) Pub. Date: Oct. 1, 2009

(54) Sanitization rack for flexible reusable substrates

(76) Inventor: Joshua David Moberg, Maple Grove, MN (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 12/080,393

(22) Filed: Apr. 1, 2008

(51) Int. Cl. A47F 5/12; A47B 81/04

(52) U.S. Cl. 211/41.8; 211/170

(57) ABSTRACT

The present invention is directed to a rack assembly comprising a plurality of support surfaces each having a planar face, wherein the planar face of each support surfaces comes into close proximity to one another such that one or more receiving voids are formed. Within the planar face of one or more of the support surfaces there are open areas which allow for the ambient environment of the rack assembly to come into fluid communication with the receiving void formed within the rack assembly. A receiving void is designed to receive a flexible reusable substrate having a front and back essentially planar surface and to maintain said flexible reusable substrate in an equivalent planar orientation of the rack assembly such that fluid communication with the ambient environment of the rack assembly will come into direct contact with a corresponding area of a flexible reusable substrate contained therein. The rack assembly is suitably tolerant to elevated temperature such that the rack assembly may be loaded with a flexible reusable substrate and positioned within an elevated temperature generating appliance and subjected to an elevated temperature treatment cycle. Suitable elevated temperature generating appliances include dish washers and microwave ovens. Upon conclusion of the elevated temperature treatment cycle, the rack assembly may be opened and the treated flexible reusable substrate removed for use. Due to exposure of the flexible reusable substrate in the rack assembly to elevated temperature cycle, the reusable substrate exhibits an increased level of cleanliness and a decreased level of potentially harmful bioburden.

### PARENT REFERENCE

Not Applicable

### BACKGROUND

Significant annual levels of illness and death are caused through contamination of food through contact with food preparation surfaces. Bacterial organisms are a primary cause of food contamination, followed by infection caused by viruses, molds, and fungi, which collectively form the bioburden routinely encountered in food preparation areas. Per a 2001 report by the Center for Disease Control, 5,000 deaths, 325,000 hospitalizations and 76 million illnesses are attributed to food poisoning per year, an increase of approximately 34% since 11948.

A number of products are commercially available to combat bioburden and thereby decrease the potential of contaminating prepared foods. These products predominantly focus on chemical means for achieving minimally biostatic conditions, if not more effective biocidal environments. While such chemistries are effective, there remains a common weakness in application of such sanitization chemistries in that a flexible substrate must be employed to distribute and/or retain the sanitization chemistries upon the targeted surfaces and to remove the chemistries post-application. Flexible substrates suitable for such distribution and removal of chemical sanitization chemistries include reusable materials such as sponges, washcloths and towels and consumable or disposable one-time use materials such as nonwoven fabric wipes. Flexible reusable substrates are particularly preferred for cleaning surfaces, particularly in residential kitchens, due to the deduced life-time cost in repeatedly using such substrate and the significant reduction in waste produced by not using a disposable substrate.

Due to the reusable nature of a flexible reusable substrate, there can arise and issue wherein the flexible reusable substrate becomes a reservoir for bioburden development and propagation. Ineffective application of sanitization chemistries, protracted use between cleanings of the substrate itself and accumulation of resilient infectious agents such as spores, viral capsules and bactericidal-resistance bacteria can result in bioburden growth within the flexible reusable substrate itself. Formation of a bioburden reservoir within the flexible reusable substrate increases the potential of contaminating otherwise clean surfaces and thereby increases the potential of inducing infection through consumption of food stuffs which may come in contact with a contaminated surface.

Numerous means and methods have been described for aiding in the sterilization of a potentially contaminated flexible reusable substrate, particularly through the use of a microwave oven. U.S. Published Patent Application No. 20030072674 to Melker et al., is directed to a sterilizer wherein a substrate is loaded into a closure formed to create a sealed high-temperature environment upon exposure to microwave energy. Japanese Published Patent Application No. 2006034645 to Oyama, is directed to a circular closure which then, similar to Melker et al., relies on a sealed high-temperature environment created by heating in a microwave oven. U.S. Pat. No. 6,270,275 to Martz utilizes a supplemental sanitization liquid chemistry dosed by the device into a sponge and subsequent elevated temperature through application of microwave energy.

Each of the aforementioned means offer a way for increasing the cleanliness of a reusable flexible substrate. However, there remains an unmet need for a further simplified means for reducing bioburden load in a reusable flexible substrate, which can be used in conjunction with multiple devices capable of creating biocidal environments, and which does not in and of itself create a hazard to the user through venting of a high temperature, pressurized device and the scald hazard associated therewith.

### SUMMARY OF THE INVENTION

The present invention is directed to a rack assembly comprising a plurality of support surfaces each having a planar face, wherein the planar face of each support surfaces comes into close proximity to one another such that one or more receiving voids are formed. Within the planar face of one or more of the support surfaces there are open areas which allow for the ambient environment of the rack assembly to come into fluid communication with the receiving void formed within the rack assembly. A receiving void is designed to receive a flexible reusable substrate having a front and back essentially planar surface and to maintain said flexible reusable substrate in an equivalent planar orientation of the rack assembly such that fluid communication with the ambient environment of the rack assembly will come into direct contact with a corresponding area of a flexible reusable substrate contained therein.

A preferred embodiment of the rack assembly includes a rack assembly being sized so as to readily fit within both the interior confines of a microwave oven and of a conventional dishwasher. The rack assembly includes a first support surface and a second support surface, wherein the first and second support surfaces include at least one hinge element and one clasp element which act upon the two support surfaces to maintain them in temporary close proximity. When the first support surface and the second support surface are in temporary close proximity through the action of the hinge element and clasp element, there is defined a receiving void between the first support surface and second support surface. The receiving void is configured to maintain at least one flexible reusable substrate positioned therein in an open and essentially planar orientation.

Alternate embodiments of the rack assembly include:
a) first and second support surface that combine to form a rack assembly having a simple contoured profile versus a planar profile;
b) first and second support surface that combine to form a rack assembly having a compound contoured profile versus a planar profile or simple contoured profile;
c) a second support surface whereby support legs extend from the second support surface; and
d) combinations of the above.

The support surface of at least one support surface includes open areas or “windows” which expose the receiving void, and subsequently a flexible reusable substrate contained therein, to the ambient environment of the overall rack assembly. Open areas in a support surface may constitute at least 30% of the total surface area available on said support surface and preferably more than at least 50% of the total surface area, and most preferably more than at least 80% of the total surface area. It is within the purview of the present invention that more than one surface support may have open areas defined therein and that in the event two or more such support surface have open areas defined therein, that the open area of any two or more support surface may be the same or different in comparison to one another.

The rack assembly is suitably tolerant to elevated temperature such that the rack assembly may be loaded with a flexible reusable substrate and positioned within an elevated temperature generating appliance and subjected to an elevated temperature treatment cycle. Suitable elevated temperature generating appliances include dish washers and microwave ovens. Dish washer treatment cycles may include high temperature water, high pressure water, repeated rinsing, exposure to detergents or chemical sanitizers and combinations thereof. Microwave oven treatment cycles include differing time period of heating, level of microwave energy used, manual wetting by an operator, and the combinations thereof. Upon conclusion of the elevated temperature treatment cycle, the rack assembly may be opened and the treated flexible reusable substrate removed for use.

Due to exposure of the flexible reusable substrate in the rack assembly to elevated temperature cycle, the reusable substrate exhibits an increased level of cleanliness and a decreased level of potentially harmful bioburden.

Other features and advantages of the present invention will become readily apparent from the following detailed description, the accompanying drawings, and the appended claims.

### BRIEF SUMMARY OF THE FIGURES

The invention will be more easily understood by a detailed explanation of the invention including drawings. Accordingly, drawings which are particularly suited for explaining the inventions are attached herewith; however, it should be understood that such drawings are for descriptive purposes only and as thus are not necessarily to scale beyond the measurements provided. The drawings are briefly described as follows:

FIG. 1 is a perspective view of a representative embodiment sanitization rack assembly in accordance with the present invention, wherein the rack assembly utilizes a first support surface and a second support surface, each said support surface having an open surface area exposing an internal receiving void and optional inclusion of support legs.

FIG. 2 is a perspective view sanitization rack as depicted in FIG. 1 wherein a hinge element allows the first support surface and second support surface to swing upon for accessing of the internal receiving void.

FIG. 3 is a top-down view of a sanitization rack.

FIG. 4 is a bottom-up view of a sanitization rack.

FIG. 5 is a left side view of a sanitization rack.

FIG. 6 is a right side view of a sanitization rack.

FIG. 7 is a back or hinge side view of a sanitization rack.

FIG. 8 is front side view of a sanitization rack.

FIG. 9 is a diagrammatic view of cross-sectional profiles the receiving void may be shaped into including straight, angled and radiused portions and the combinations thereof.

FIG. 10 is a schematic representation of how a sanitization rack in accordance with the present invention may be used for the sanitization of a typical household cleaning sponge.

### DETAILED DESCRIPTION

While the present invention is susceptible of embodiment in various forms, there is shown in the drawings and will hereinafter be described a presently preferred embodiment of the invention, with the understanding that the present disclosure is to be considered as an exemplification of the invention, and is not intended to limit the invention to the specific embodiment illustrated.

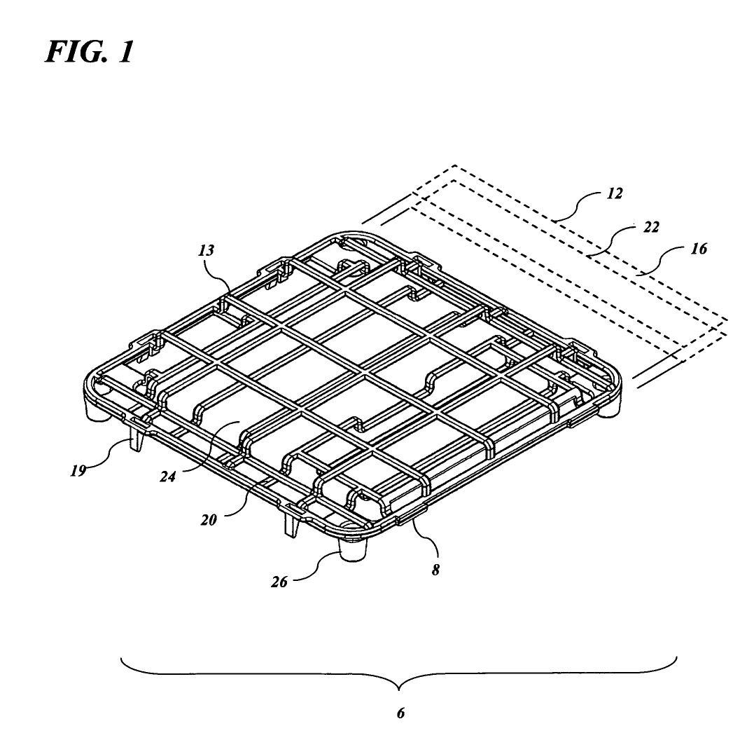

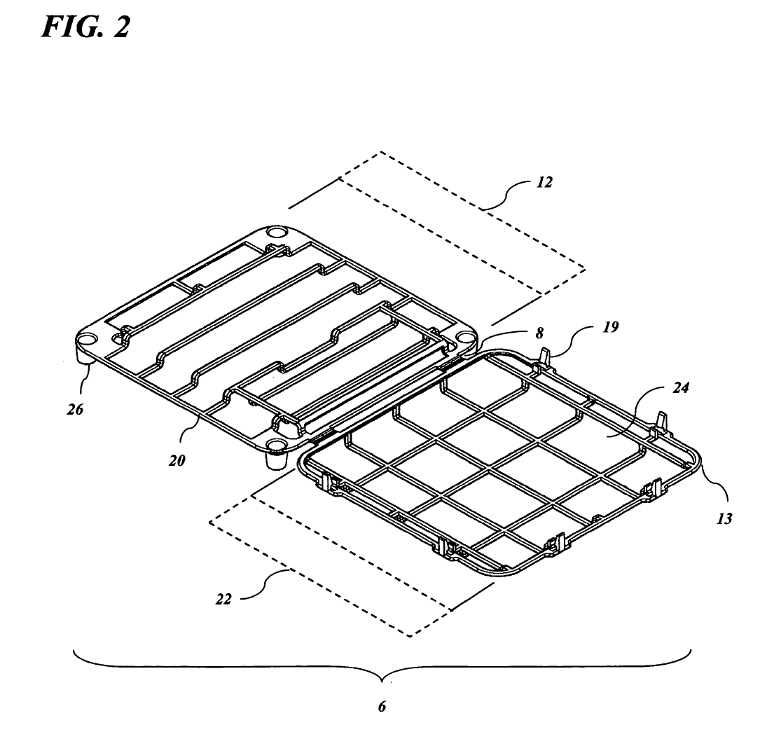

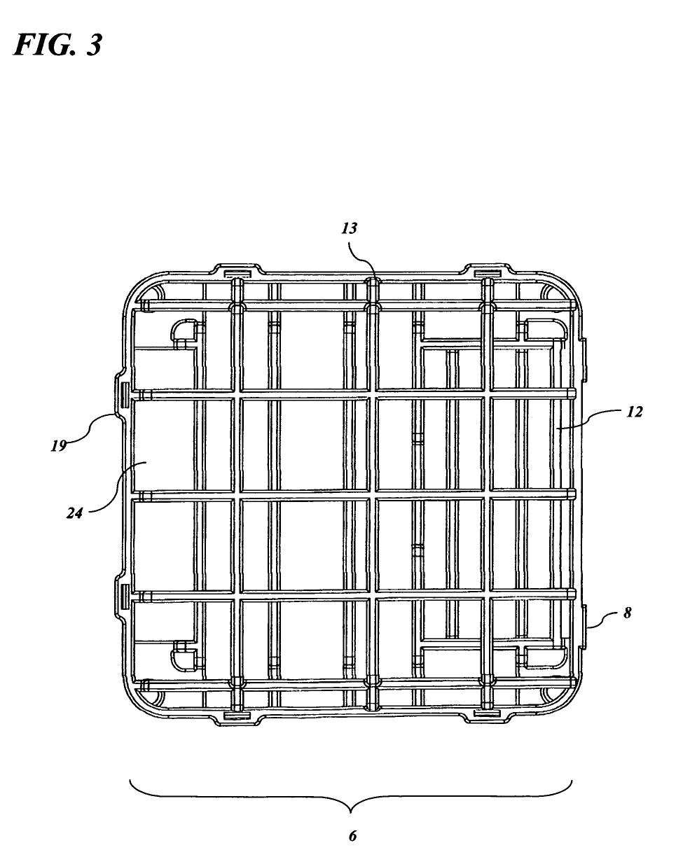

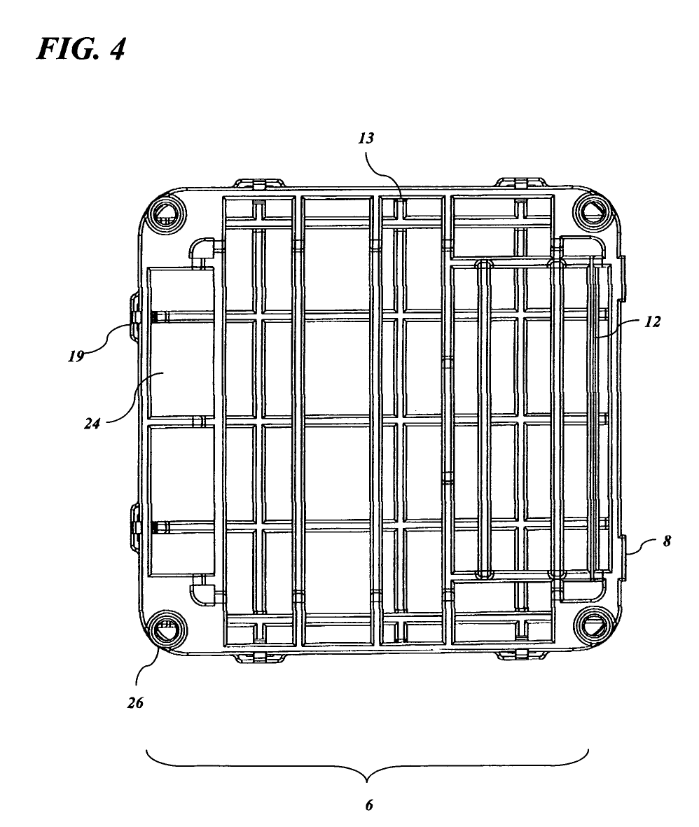

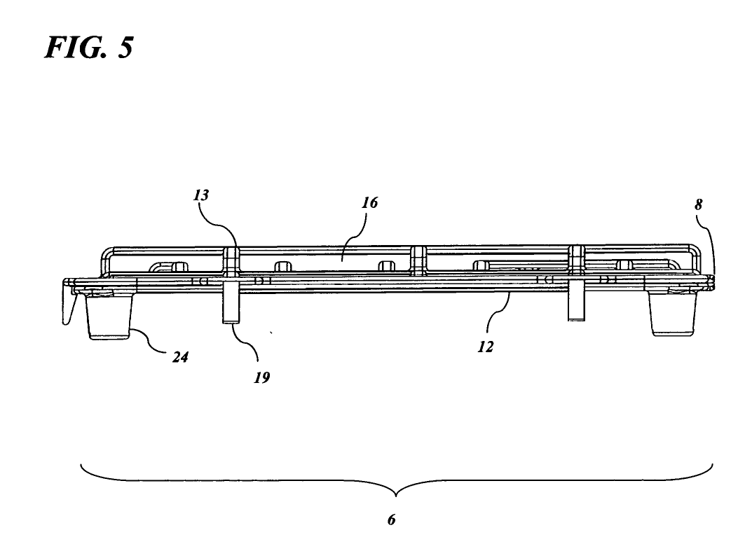

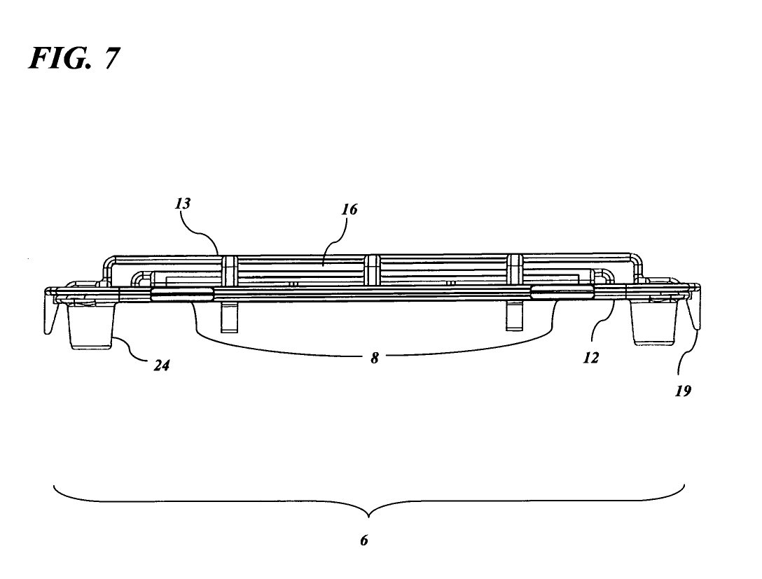

Referring more specifically to FIGS. 1 through 8, there is shown a rack assembly 6 comprising a first support surface 13 and a second support surface 20. First support surface 13 has an essentially planar face defined by plane 12 and second support surface 20 has an essentially planar face defined by plane 22. In the disclosed first embodiment, first support surface 13 and second support surface 20 are closely positioned so that planar face 12 and planar face 22 come into proximity at a closest point of less than 0.75 inch to one another, preferably less than 0.50 inch and most preferably 0.35 inch. Defined by between planar face 12 and planar face 22 is a gap or space which constitutes receiving void 16.

Within planar face 12 and planar face 22 there are open areas 24. Open areas 24 extend completely through the respective support surface and form “windows” which expose receiving void 16, and subsequently a flexible reusable substrate 40 contained therein, to the ambient environment of the overall rack assembly. Open areas 24 in a support surface may constitute at least 30% of the total surface area available on said support surface and preferably more than at least 50% of the total surface area, and most preferably more than at least 80% of the total surface area. It is within the purview of the present invention that more than one surface support may have open areas defined therein and that in the event two or more such support surface have open areas defined therein, that the open area of any two or more support surface may be the same or different in comparison to one another.

Flexible reusable substrate 40 can include reusable materials such as sponges, washcloths and towels. Representative dimensions for flexible reusable substrate 40 include widths of 2.5 inches to 12 inches, heights of 3.0 inches to 12 inches, and depths of 0.13 inch to 0.75 inch.

Receiving void 16 is designed to receive at least one flexible reusable substrate 40 having a front and back essentially planar surface and to maintain said flexible reusable substrate 40 in an equivalent planar orientation of the rack assembly such that fluid communication with the ambient environment of the rack assembly will come into direct contact with a corresponding area of flexible reusable substrate 40 contained therein. The maintenance of flexible reusable substrate 40 an open and essentially planar orientation is important in ensuring improved uniformity of cleanliness by avoiding creation within the substrate of inaccessible areas to cleaning action of the ambient environment or preventing exposure to elevated temperatures, such as may be caused folding or bunching of the flexible reusable substrate upon itself. It may be desirable to further incorporate friction modifying asperities or durable friction chemistries to one or more of the support surfaces exposed to the receiving void to aid in maintaining the flexible reusable substrate in an open orientation.

Rack assembly 6 including first support surface 13 and second support surface 20, further includes at least one hinge element 8 and one or more clasp element 19 which act upon the first support surfaces 13 and second support surface 20 to maintain them in temporary close proximity. Hinge element 8 may include any suitable means which allows for rotation of first support surface 13 and second support surface about a common axis of at least 190°. Representative means for creating hinge element 8 includes: use of at least two interdigitated hinge plates each extending from a respective support surface where an axle or pin links the hinge plates to allow for rotation; use of one or more circular rings which extend perpendicular to and around an outer edge of each support surface; and, linking the first support surface to the second support surface at the time on manufacture with a continuous pliable plastic strip so as to create a living hinge. One or more clasp element 19 may include any suitable means which allows for releasable engagement by a user of first support surface 13 from second support surface to then allow rotation about a common hinge element 8. Representative means for creating clasp element 19 includes: interlocking keyways, tab and barb, camming locks, sliding lock with notch, and hinged snaps. In the event more than one clasp element 19 is employed, more than one type of releasable engage means may be used.

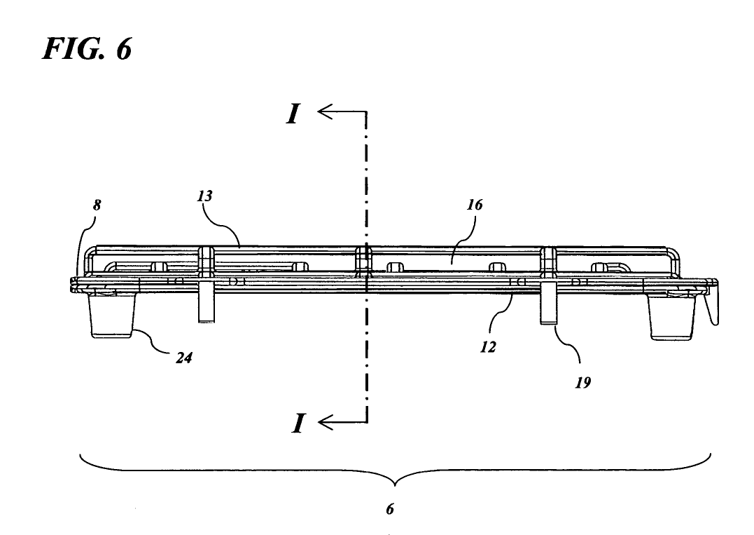

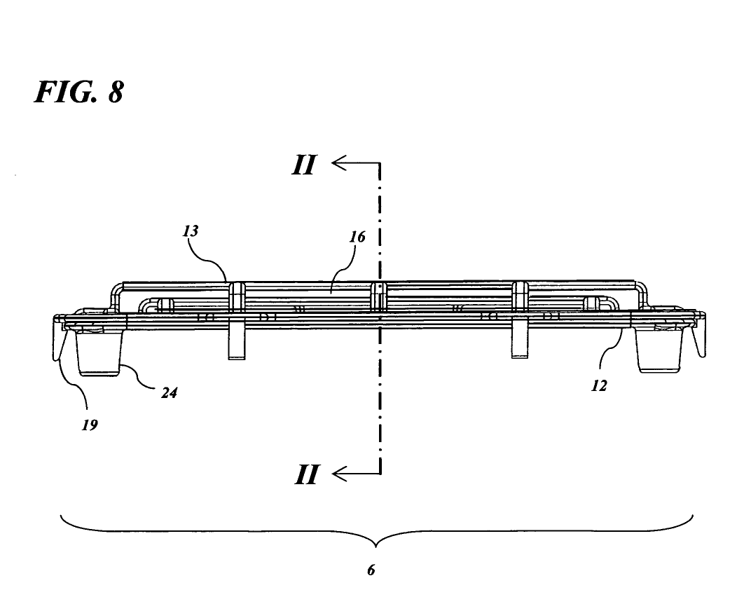

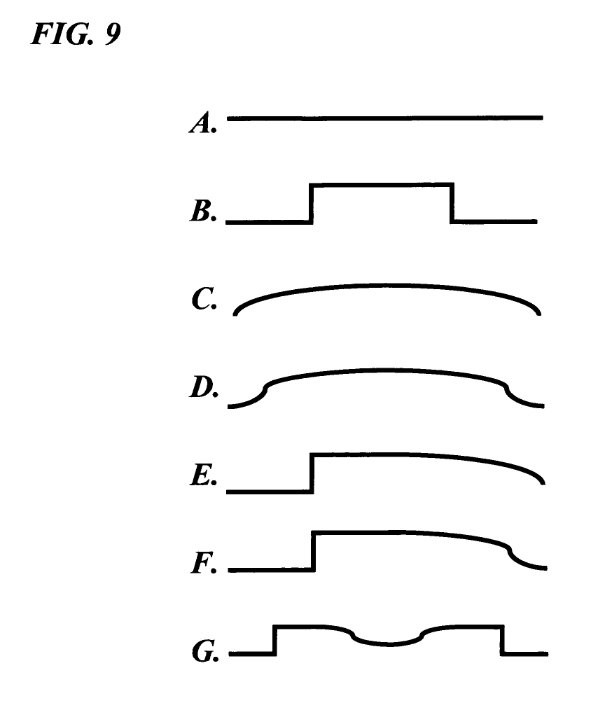

Referring to FIG. 9, therein is depicted alternate embodiments of the present invention whereby the first and second support surfaces have a contoured profile and maintain an essentially parallel and close proximity to one another to define a receiving area of varying shape. The representative profiles shown in FIG. 9 may correspond to the general cross section profile of the first support surface 13, the second support surface 20, or the receiving void 16. Cross sectional profiles in FIG. 9 may be taken at line I-I in FIG. 6, at line II-II in FIG. 8, or in combination of both line I-I in FIG. 6 and line II-II in FIG. 8. The benefit in creating a contoured profile is that at least part of the rack assembly is raised up an away from a base region the surface area of which would have otherwise been occupied by the rack assembly itself. This raised aspect of the rack assembly can be beneficial in allowing better flow of the fluidic elevated temperature environment around and into the rack assembly.

Referring to FIGS. 1 through 8, therein is depicted an optional element whereby the second support surface has a plurality of supporting legs 26. The benefit in use of supporting legs 26 is that at least part of the rack assembly is raised up an away from a base region the surface area of which would have otherwise been occupied by the rack assembly itself. This raised aspect of rack assembly 6 can be beneficial in allowing better flow of the fluidic elevated temperature environment around and into the rack assembly.

The components used in the fabrication of rack assembly 6 should be suitably tolerant to elevated temperature such that the rack assembly may be loaded with a flexible reusable substrate and positioned within an elevated temperature generating appliance and subjected to an elevated temperature treatment cycle without undue deformation or warping. A preferred mode of fabrication is through practice of injection molding technologies using plastic resins loaded under high pressure into a mould having the inverse topography of the component desired. Ideally, the plastic resins employed will be resistant to expected exposure temperatures, with a safety margin of at least 15% between the expected maximum exposed temperature and the softening point of the resin. Further, plastic resins that are transparent and/or non-reactive to microwave energy are desirable. For the purposes of providing representative plastic resins appropriate for manufacturing a sanitization rack in accordance with the present invention, the inventors suggest polyolefins, nylons, polyesters, and polyvinyls. It is within the purview of the present invention that aesthetic and performance modifying admixtures and chemistries can be combined with the base plastic resin so as to achieve attributes beyond those inherent to the base resin chemistry itself.

Elevated temperature generating appliances appropriate for use with a sanitization rack containing a flexible reusable substrate include common dishwashers and microwave ovens. Dish washer treatment cycles may include high temperature water, high pressure water, repeated rinsing, exposure to detergents or chemical sanitizers and combinations thereof which are normally employed when cleaning flatware, stoneware and glass. Microwave oven treatment cycles include differing time period of heating, level of microwave energy used, manual wetting by an operator, and the combinations thereof. Upon conclusion of the elevated temperature treatment cycle, the rack assembly may be opened and the freshly treated flexible reusable substrate removed for use.

High temperatures are useful in creating biocidal environments through a number of different modalities; including denaturation, lysis and desiccation. Denaturation events include loss of form, and consequently functionality, of organelles, proteins and nucleic acids necessary for the continued respiration and/or viability of an organism. Lysis events include destruction of cellular membranes and walls of an organism which protect and maintain necessary internal chemical environments for that organism to remain viable. Desiccation, particular at very high temperatures, for prolonged periods of time or through exposure to high energy, is deleterious to organism through osmotic failure and collapse of biochemical pathways. The ambient environment created by a common dishwasher is useful in reducing bioburden load through the combined thermal, mechanical and chemical action of the appliance. Microwave ovens are equally effective at inducing cellular death, though there is no mechanical or chemical action as per the use of a dishwasher, the high energy microwaves are particularly destructive.

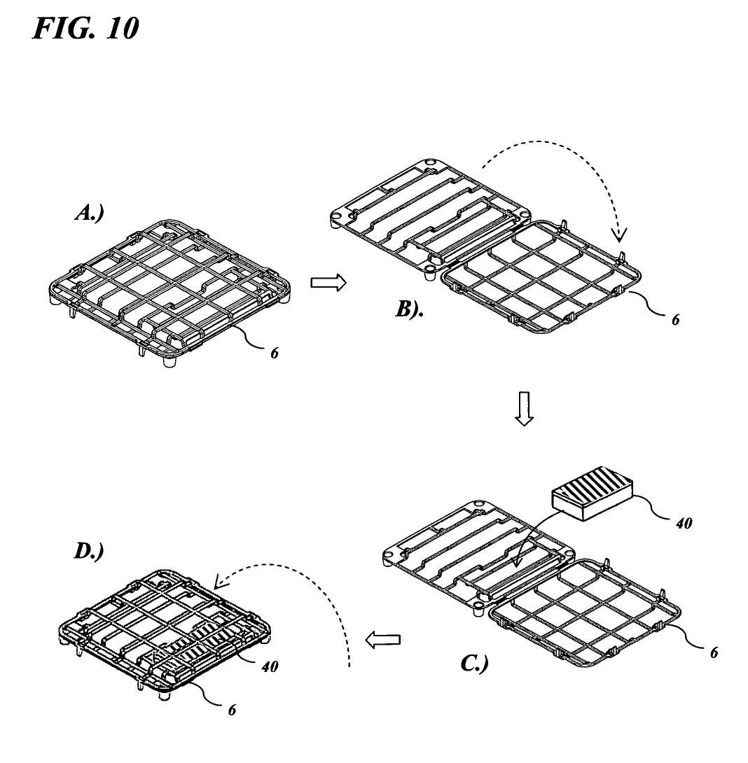

FIG. 10 depicts a typical loading procedure for a rack assembly 6 with a flexible reusable substrate 40. One or more clasp elements are manually released by the user and the first support surface is swung away from the second support surface about the hinge (FIG. 10-B). A flexible reusable substrate 40 (e.g. a common houseful cleaning sponge) is placed on the second support surface (FIG. 10-C). The first support surface is swung back into place atop second support surface, thereby capturing the flexible reusable substrate within a receiving void created between first support surface and second support surface wherein the clasp elements are reengaged to maintain the assembly in a closed state (FIG. 10-D). As can be noted in FIG. 10, the flexible reusable substrate is exposed to the exterior environment of rack assembly 6. When rack assembly 6 with flexible reusable substrate captured within is then placed into a suitable elevated temperature environment (such as a dish washing machine or microwave oven, not shown) the elevated temperatures generated can act upon the flexible reusable substrate and thereby cause reduction in the bioburden associated therewith.

From the foregoing, it will be observed that numerous modifications and variations can be affected without departing from the true spirit and scope of the novel concept of the present invention. It is to be understood that no limitation with respect to the specific embodiments illustrated herein is intended or should be inferred. The disclosure is intended to cover, by the appended claims, all such modifications as fall within the scope of the claims.

1. A sanitization rack comprising of:
a. a first support surface;
b. a second support surface;
c. a hinge element connecting said first support surface and said second support surface and allowing said support surfaces to open at least 190° relative to one another;
d. a clasp element affixed to said first support surface and capable of being releasably affixed to said second support surface;
e. a receiving void formed between said first support surface and said second support surface when said hinge element is at 0° and said clasp element is reliably affixed to said second support surface;
wherein said first support surface has an open area defined through said first support surface; and
wherein a flexible reusable substrate is placed with said receiving void.

2. A sanitization rack as in claim 1, wherein said open area of said first support surface is at least 30% of an overall surface area within said first support surface.

3. A sanitization rack as in claim 1, wherein said open area of said first support surface is at least 50% of an overall surface area within said first support surface.

4. A sanitization rack as in claim 1, wherein said open area of said first support surface is at least 80% of an overall surface area within said first support surface.

5. A sanitization rack as in claim 1, wherein said second support surface has an open area defined through said first support surface.

6. A sanitization rack as in claim 5, wherein said open area of said second support surface is at least 30% of an overall surface area within said first support surface.

7. A sanitization rack as in claim 1, wherein said open area of said second support surface is at least 50% of an overall surface area within said first support surface.

8. A sanitization rack as in claim 1, wherein said open area of said second support surface is at least 80% of an overall surface area within said first support surface.

9. A method for sanitizing a flexible reusable substrate, comprising of a sanitization rack, a flexible reusable substrate and an elevated temperature environment, wherein said sanitization rack comprising of:
a. a first support surface;
b. a second support surface;
c. a hinge element connecting said first support surface and said second support surface and allowing said support surfaces to open at least 90° relative to one another;
d. a clasp element affixed to said first support surface and capable of being releasably affixed to said second support surface;
e. a receiving void formed between said first support surface and said second support surface when said hinge element is at 0° and said clasp element is reliably affixed to said second support surface;
wherein said first support surface has an open area defined through said first support surface;
wherein said flexible reusable substrate is placed with said receiving void of said sanitization rack; and
wherein said sanitization rack containing said flexible reusable substrate is placed into said elevated temperature environment and said elevated temperature environment is in direct fluid contact with said flexible reusable substrate through said open area of said first support surface.

10. A method for sanitizing a flexible reusable substrate as in claim 9, wherein said open area of said first support surface is at least 30% of an overall surface area within said first support surface.

11. A method for sanitizing a flexible reusable substrate as in claim 9, wherein said open area of said first support surface is at least 50% of an overall surface area within said first support surface.

12. A method for sanitizing a flexible reusable substrate as in claim 9, wherein said open area of said first support surface is at least 80% of an overall surface area within said first support surface.

13. A method for sanitizing a flexible reusable substrate as in claim 9, wherein said second support surface has an open area defined through said first support surface.

14. A method for sanitizing a flexible reusable substrate as in claim 13, wherein said open area of said second support surface is at least 30% of an overall surface area within said first support surface.

15. A method for sanitizing a flexible reusable substrate as in claim 13, wherein said open area of said second support surface is at least 50% of an overall surface area within said first support surface.

16. A method for sanitizing a flexible reusable substrate as in claim 13, wherein said open area of said second support surface is at least 80% of an overall surface area within said first support surface.

17. A method for sanitizing a flexible reusable substrate as in claim 9, wherein said elevated temperature environment is created by a dishwasher.

18. A method for sanitizing a flexible reusable substrate as in claim 9, wherein said elevated temperature environment is created by a microwave oven.
